(19) United States
(12) Patent Application Publication
(10) Pub. No.: US 2020/0060930 A1
(43) Pub. Date: Feb. 27, 2020

(54) COMPOSITE AND AN ARTICLE COMPRISING THE SAME

(71) Applicant: TAI YU ARTS & DEVELOPMENT CO., LTD., Chiayi City (TW)

(72) Inventors: MING CHING LU, TAIPEI (TW); MAO TING YANG, CHIAYI CITY (TW)

(21) Appl. No.: 16/394,486

(22) Filed: Apr. 25, 2019

(30) Foreign Application Priority Data
Aug. 24, 2018 (TW) 107129598

(51) Int. Cl. A61H 39/00; B32B 27/20; B32B 27/38; B32B 27/30; B32B 27/40; A43B 17/00; A43B 7/00; A61L 31/12; A61L 31/14

(52) U.S. Cl. CPC A61H 39/007; A61H 2201/165; B32B 27/38; B32B 27/308; B32B 27/40; A43B 17/006; A43B 7/005; A61L 31/122; A61L 31/14; B32B 2262/106; B32B 2307/30; B32B 2437/00; B32B 2437/02; A61L 2400/00; A61H 2201/1253; A61H 2201/1642; B32B 27/20

(57) ABSTRACT

The present invention relates to composites for reflecting energy produced from a body to the body itself. Accordingly, the present invention provides composites comprising inert materials and methods of using the composites to enhance health conditions of animals by reflecting the energy produced from a body to the body itself.

### FIELD OF THE INVENTION

The present invention is directed to a composite for reflecting energy produced from a body to the body itself. Particularly, the present invention pertains to a substrate comprising inert materials and methods of using the composites to enhance health conditions of animals by reflecting the energy produced from a body to the body itself.

### BACKGROUND OF THE INVENTION

Physical therapy (physiotherapy), a complementary and alternative medicine therapy, has been widely applied in diagnosing and treating various diseases and defects. Physiotherapy can help people avoid surgery, reduce medications, manage pain and reduce the risk of injury. Physical therapy uses mechanical force and movements (bio-mechanics or kinesiology), manual therapy, exercise therapy, and electrotherapy, remediates impairments and promotes mobility and function.

U.S. Pat. No. 5,451,199 provides a portable device for carrying with the human body that includes a far infrared ray generating composition plate, a magnetic radiating unit, an electronic circuit unit, and a mercury battery disposed on the far infrared ray generating composition plate for self-radiating bioenergy so as to eliminate and reduce human fatigue and stress. U.S. Pat. No. 6,461,377 relates to a portable therapeutic device that radiates far infrared rays from Macbanseok received therein and applies electric stimulation to the body by means of an electric needle, in addition to generating magnetic vibration through rotation of two permanent magnets having magnetic polarities different from each other. U.S. Pat. No. 6,516,229 describes a personal therapeutic device comprising a supporting means with a far infrared ray emitting body placed thereon that provides a means for enhancing the health conditions of human beings. U.S. Pat. No. 6,860,896 provides an energy therapy device that utilizes an array of energy-emitting elements to stimulate Qi energy flow along acupuncture meridians.

However, there is a need to develop an article for enhancing health conditions of a human by physical therapy.

### SUMMARY OF THE INVENTION

The present disclosure provides a method of enhancing health of a body, comprising applying a composite comprising inert materials to the body, whereby the composite can reflect energy produced by the body to the body itself so as to enhance health conditions of the body.

In some embodiments, the composite is attached to or enclosed in an article. In some embodiments, the article is a patch, shoe pad, belt, bust pad, abdominal pad, neck strap, bend, tank top, vest, hat, or underwear.

In one aspect, the present disclosure provides a composite comprising a first layer and a second layer attached on the first layer, wherein the first layer comprises carbon fiber in the amount of about 65% to about 90% (w/w) based on the total composite and the second layer comprising, based on the total composite, graphene in an amount of about 0.01% to about 1% (w/w), carbon nanotube in the amount of 0.1% to about 3.0% (w/w), and a resin or an adhesive material in an amount of about 9.9% to about 35% (w/w).

In another aspect, the present disclosure provides a laminar, comprising two or more composites of the present disclosure.

### DETAILED DESCRIPTION OF THE INVENTION

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

As used herein and in the appended claims, the singular forms “a,” “and,” and “the” include plural referents unless the context clearly dictates otherwise.

Also, the use of “or” means “and/or” unless stated otherwise. Similarly, “comprise,” “comprises,” “comprising” “include,” “includes,” and “including” are interchangeable and not intended to be limiting.

When the body breaks down food molecules, the energy in the chemical bonds of the food is released, which powers the body. However, the human body is only about 25 percent efficient, meaning that 75 percent of the energy from the food is released as heat. Heat transfer through radiation takes place in the form of electromagnetic waves mainly in the infrared region. Radiation emitted by a body is a consequence of thermal agitation of its composing molecules. Accordingly, the present disclosure is based on a composite comprising inert materials and an article having the composite, which can reflect energy produced by a body to the body itself so as to enhance health conditions of the body.

The composite of the present disclosure comprises inert materials such as graphene, carbon nanotubes, carbon fibers and resins. The composite per se of the present disclosure does not emit energy but can reflect energy produced by a body to the body itself so as to enhance health conditions of the body. In one embodiment, the energy to be reflected by the composite is an electromagnetic wave having wavelength of about 4 μm to about 14 μm. The wavelength of about 4 μm to about 14 μm falls within the region of far infrared (FIR). Objects with temperatures between about 5 K and 340 K will emit radiation in the far infrared range. Thermal radiation, also known as heat, is the emission of electromagnetic waves from all matter that has a temperature greater than absolute zero. The composite of the present disclosure reflects the electromagnetic wave emitted from human body.

In one embodiment, the present disclosure provides a composite comprising a first layer and a second layer attached on the first layer, wherein the first layer comprises carbon fiber in the amount of about 65% to about 90% (w/w) based on the total composite and the second layer comprising, based on the total composite, graphene in an amount of about 0.01% to about 1% (w/w), carbon nanotube in the amount of 0.1% to about 3.0% (w/w), and a resin or an adhesive material in an amount of about 10% to about 35% (w/w).

Graphene is a monoatomic layer of carbon atoms, sp² hybridized, organized according to a crystalline structure with hexagonal cells. This basic structure has a planar conformation and consequently the monoatomic layer appears as a two-dimensional material. Due to the particular geometry of the system and electronic configuration of carbon, graphene has a very specific electronic band structure, with exceptional mechanical, and also electronic, characteristics. With a Young modulus equal to 1 TPa and a tensile strength equal to 130 GPa, graphene is the most resistant material so far measured. Furthermore, it has a thermal conductivity of 5,000 W/mK and electrical resistivity of 4×10⁻⁵ Ohm·cm.

The carbon nanotubes exhibit an insulator, a conductor or a semiconductor property depending on the chirality of the tube itself. Carbon nanotubes have strong tensile strength more than 100 times greater than steel due to their strong covalent bond with carbon atoms, excellent flexibility and elasticity, and chemically stable characteristics. Due to such size and specific physical properties, they are industrially important in the production of composites and have high utility in the fields of electronic materials, energy materials and various other fields.

Carbon fibers are low-cost reinforced materials which are light and strong, and have a high modulus of elasticity. Carbon fibers are fibers about 5-10 micrometers in diameter and composed mostly of carbon atoms. Carbon fibers have several advantages including high stiffness, high tensile strength, low weight, high chemical resistance, high temperature tolerance and low thermal expansion.

Resin is a solid or highly viscous substance of plant or synthetic origin that is typically convertible into polymers. Resins are usually mixtures of organic compounds. In one embodiment, the resin is thermoplastic resin. In some embodiments, the resin includes, but is not limited to, polyethylene (PE), polypropylene (PP), polyester, thermoplastic polyurethane (TPU), thermoplastic elastomer (TPE), acrylic resin, polyurethane (PU), silicone resin, polyvinyl chloride (PVC), polystyrene (PS), polyamide (PA), polyoxymethylene (POM), polycarbonate (PC), poly(methyl methacrylate) (PMMA), epoxy, thermoplastic polyurethane (TPU) and thermoplastic rubber (TPR) and any combinations thereof. In some embodiments, the resin is epoxy, PMMA or TPU.

An adhesive material can be used to fix the resin, graphene and carbon nanotube and adhere the second layer to the first layer. In one embodiment, the resin can be used as the adhesive material. In some embodiments, the adhesive material includes, but is not limited to, PMMA, or polyurethane, epoxy and any modified form thereof.

In one embodiment, the amount of carbon fiber ranges from about 65% to about 90% (w/w), about 65% to about 80% (w/w), about 65% to about 70% (w/w), about 70% to about 90% (w/w) or about 70% to about 80% (w/w).

In one embodiment, the amount of graphene ranges from about 0.01% to about 1% (w/w), about 0.05% to about 1% (w/w), about 0.1% to about 1% (w/w), about 0.5% to about 1% (w/w), about 0.01% to about 0.5% (w/w), about 0.01% to about 0.1% (w/w) or about 0.01% to about 0.05% (w/w).

In one embodiment, the amount of carbon nanotube ranges from about 0.1% to about 3.0% (w/w), about 0.1% to about 2.5% (w/w), about 0.1% to about 2.0% (w/w), about 0.1% to about 1.5% (w/w), about 0.1% to about 1.0% (w/w), about 0.1% to about 0.5% (w/w), about 0.5% to about 3.0% (w/w), about 0.5% to about 2.5% (w/w), about 0.5% to about 2.0% (w/w), about 0.5% to about 1.5% (w/w), about 0.5% to about 1.0% (w/w), about 1.0% to about 3.0% (w/w), about 1.0% to about 2.5% (w/w), about 1.0% to about 2.0% (w/w), about 1.0% to about 1.5% (w/w), about 1.5% to about 3.0% (w/w), about 1.5% to about 2.5% (w/w), about 1.5% to about 2.0% (w/w), about 2.0% to about 3% (w/w) or about 2.5% to about 3% (w/w).

In one embodiment, the amount of resin or an adhesive material ranges from about 10% to about 35% (w/w), about 10% to about 30% (w/w), about 10% to about 25% (w/w), about 10% to about 20% (w/w), about 10% to about 15% (w/w), about 15% to about 35% (w/w), about 15% to about 30% (w/w) or about 15% to about 25% (w/w), about 20% to about 35% (w/w), about 20% to about 30% (w/w).

In one embodiment, the first layer and the second layer can be in any thickness based on the user's need.

The present disclosure provides a laminate comprising two or more composites of the present disclosure.

The present disclosure also provides an article comprising a composite of the present disclosure attached to or enclosed in the article. In some embodiments, the article is a patch, shoe pad, belt, bust pad, abdominal pad, neck strap, bend, tank top, vest, hat, or underwear. The article can be in any shape.

The present disclosure also provides a method of enhancing health of a body, comprising applying a composite of the present disclosure comprising inert materials to the body, whereby the a composite of the present disclosure can reflect energy produced by the body to the body itself so as to enhance health conditions of the body.

In one embodiment, the composite is the one described herein.

Example embodiments may, however, be embodied in many different forms and should not be construed as being limited to the embodiments set forth herein; rather, these example embodiments are provided so that this disclosure will be thorough and complete, and will fully convey the scope of example embodiments of the present disclosure to those of ordinary skill in the art.

### EXAMPLES

The composites of the present disclosure having the following formulations were enclosed in shoe pads and meridian patches.

| | First layer | Second layer |
|---|---|---|
| Composite A | 70% (w/w) | 1% (w/w) graphene, 0.1% |
| | carbon fiber | (w/w) carbon nanotube, |
| | | 28.9% (w/w) epoxy |
| Composite A | 80% (w/w) | 0.05% (w/w) graphene, 2% |
| | carbon fiber | (w/w) carbon nanotube, |
| | | 17.95% (w/w) PMMA |
| Composite C | 90% (w/w) | 0.01% (w/w) graphene, 1% |
| | carbon fiber | (w/w) carbon nanotube, |
| | | 8.99% (w/w) TPU |

Shoe Pad Testing

Six men and six women wore shoes with the shoe pads enclosed with Composite A, B or C for 8 hours per day for one week. After the testing, the following results detected by an AE Organism Electric Analyzer were obtained:

Improvement on vascular resistance: more than 5%;
Improvement on vascular elasticity: more than 10%;
Improvement on cerebrovascular elasticity: more than 10%;
Improvement on hyperosteogeny: more than 10%; and
Improvement on osteoporosis: more than 10%.

Meridian Patch Testing

Six men and six women attended the testing. The meridian patches enclosed with Composite A, B or C were applied to yongquan acupoint on soles of the subjects for 8 hours per day for one week. Then, the subjects conducted a grip strength testing and the results measured by Hand Dynamometer were shown in the table below.

| | | | Grip Strength | |
|---|---|---|---|---|
| | | Grip Strength | (kg) on the | |
| | | (kg) on the | seventh | Improvement (%) |
| Subject No. | | first day (a) | day (b) | ((b − a)/a × 100) |
| 1 | (male) | 30.1 | 34.0 | 12.95 |
| 2 | (male) | 28.0 | 32.3 | 15.36 |
| 3 | (male) | 28.5 | 32.0 | 12.28 |
| 4 | (male) | 27.3 | 30.3 | 10.99 |
| 5 | (male) | 29.6 | 33.9 | 14.52 |
| 6 | (male) | 22.4 | 26.0 | 16.07 |
| 7 | (female) | 18.5 | 21.1 | 14.05 |
| 8 | (female) | 21.0 | 25.2 | 20.00 |
| 9 | (female) | 15.7 | 19.0 | 21.02 |
| 10 | (female) | 10.3 | 12.0 | 16.50 |
| 11 | (female) | 11.8 | 13.4 | 13.56 |
| 12 | (female) | 7.7 | 8.7 | 12.98 |
| | | | | 15.02 in average |

The subjects also conducted a balance testing for Composite A, B or C and the results were shown in the table below. A circle with a diameter of 30 cm was drawn on the ground and each subject stood in the center of the circle and turned around 10 times in circles. The results are shown in the table below.

| | | | Shift distance | |
|---|---|---|---|---|
| | | Shift distance | (cm) on the | |
| | | (cm) on the | seventh | Improvement (%) |
| Subject No. | | first day (a) | day (b) | ((b − a)/a × 100) |
| 1 | (male) | 30 | 10 | 66.7 |
| 2 | (male) | 35 | 24 | 31.4 |
| 3 | (male) | 40 | 20 | 50.0 |
| 4 | (male) | 45 | 28 | 37.8 |
| 5 | (male) | 46 | 30 | 34.8 |
| 6 | (male) | 50 | 26 | 48.0 |
| 7 | (female) | 40 | 22 | 45.0 |
| 8 | (female) | 33 | 18 | 45.4 |
| 9 | (female) | 46 | 30 | 34.8 |
| 10 | (female) | 60 | 40 | 33.3 |
| 11 | (female) | 55 | 33 | 40.0 |
| 12 | (female) | 48 | 27 | 43.7 |
| | | | | 42.6 in average |

Although the present disclosure and its advantages have been described in detail, it should be understood that various changes, substitutions and alterations can be made herein without departing from the spirit and scope of the disclosure as defined by the following claims.

What is claimed is:

1. A composite comprising a first layer and a second layer attached on the first layer, wherein the first layer comprises carbon fiber in the amount of about 65% to about 90% (w/w) based on the total composite and the second layer comprises, based on the total composite, graphene in an amount of about 0.01% to about 1% (w/w), carbon nanotube in an amount of 0.1% to about 3.0% (w/w), and a resin or an adhesive material in an amount of about 10% to about 35% (w/w).

2. The composite of claim 1, wherein the resin is polyethylene (PE), polypropylene (PP), polyester, thermoplastic polyurethane (TPU), thermoplastic elastomer (TPE), acrylic resin, polyurethane (PU), silicone resin, polyvinyl chloride (PVC), polystyrene (PS), polyamide (PA), polyoxymethylene (POM), polycarbonate (PC), poly(methyl methacrylate) (PMMA) or thermoplastic rubber (TPR) or a combination thereof.

3. The composite of claim 1, wherein the adhesive material is PMMA, polyurethane, epoxy or any modified form thereof.

4. The composite of claim 1, wherein the amount of carbon fiber ranges from about 70% to about 90% (w/w).

5. The composite of claim 1, wherein the amount of graphene ranges from about 0.05% to about 1% (w/w).

6. The composite of claim 1, wherein the amount of carbon nanotube ranges from about 0.1% to about 2.5% (w/w).

7. The composite of claim 1, wherein the amount of resin or an adhesive material ranges from about 10% to about 30% (w/w).

8. The composite of claim 1, which can reflect energy produced by vibration of water molecules within body to the body itself so as to improve human health.

9. A laminate comprising two or more composites of claim 1.

10. An article comprising a composite of claim 1 attached to or enclosed in the article.

11. The article of claim 10, which is a patch, shoe pad, belt, bust pad, abdominal pad, neck strap, bend, tank top, vest, hat, or underwear.

12. A method of enhancing health of a body, comprising applying a composite of claim 1 to the body, whereby the composite of claim 1 can reflect energy produced by the body to the body itself so as to enhance health conditions of the body.
